(19) United States
(12) Patent Application Publication
(10) Pub. No.: US 2021/0022715 A1
(43) Pub. Date: Jan. 28, 2021

(54) METHOD FOR OBJECTIVE, NONINVASIVE STAGING OF DIFFUSE LIVER DISEASE FROM ULTRASOUND SHEAR-WAVE ELASTOGRAPHY

(71) Applicants: The General Hospital Corporation, Boston, MA (US); MASSACHUSETTS INSTITUTE OF TECHNOLOGY, Boston, MA (US)

(72) Inventors: Laura Brattain, Lexington, MA (US); Manish Dhyani, Boston, MA (US); Anthony E. Samir, Newton, MA (US); Brian A. Telfer, Lincoln, MA (US)

(21) Appl. No.: 17/040,473

(22) PCT Filed: Mar. 26, 2019
(86) PCT No.: PCT/US2019/024021

§ 371 Date: Sep. 22, 2020

Related U.S. Application Data
(60) Provisional application No. 62/648,033, filed on Mar. 26, 2018.

(51) Int. Cl. A61B 8/08; A61B 8/00; G16H 50/20; G16H 30/20; G16H 30/40; G16H 15/00; G16H 50/30; G16H 50/50

(52) U.S. Cl. CPC A61B 8/5223; A61B 8/485; A61B 8/469; A61B 8/08; A61B 8/5269; G16H 50/50; G16H 30/20; G16H 30/40; G16H 15/00; G16H 50/30; G16H 50/20

(57) ABSTRACT

Systems and methods are provided for objective, noninvasive staging of diffuse liver disease, and other diseases, from ultrasound shear wave elastography (“SWE”). A liver fibrosis staging SWE image analysis algorithm or toolkit may be used to detect the presence of liver disease and may integrate demographic, clinical, laboratory, conventional sonographic, SWE data, and the like into a combined disease diagnosis decision support model, such as for the diagnosis of high risk liver disease. Advanced image processing methods may be used to extract fibrosis pattern information from SWE and ultrasound (US) image data and this can be used to augment stiffness-based liver fibrosis staging methods.

### CROSS-REFERENCE TO RELATED APPLICATIONS

This application claims the benefit of U.S. Provisional Patent Application Ser. No. 62/648,033, filed on Mar. 26, 2018, and entitled “Method for Objective, Noninvasive Staging of Diffuse Liver Disease from Ultrasound Shear-Wave Elastography,” which is herein incorporated by reference in its entirety.

### STATEMENT REGARDING FEDERALLY SPONSORED RESEARCH

This invention was made with government support under FA8702-15-D-0001 awarded by the Department of Defense, and EB020710 awarded by the National Institutes of Health. The government has certain rights in the invention.

### BACKGROUND

Diffuse liver disease, particularly non-alcoholic fatty liver disease (NAFLD), is widespread. NAFLD is exceptionally common, with an estimated one hundred million people afflicted in the United States alone. NAFLD, the most common United States chronic liver disease (CLD), is expected soon to be the leading cause of end-stage liver disease (ESLD) and the dominant reason for liver transplantation. NAFLD prevalence, currently 25-40%, has doubled in the last 20 years and continues to increase. Despite recent advances, including development of numerous therapeutic agents, NAFLD remains a silent disease in which the vast majority of patients accumulate progressive liver damage without signs or symptoms and, undiagnosed, receive no medical care. 3-8% of the US population has nonalcoholic steatohepatitis (NASH), a NAFLD subgroup characterized by hepatocellular injury, which can progress to ESLD and hepatocellular carcinoma.

NAFLD, NASH, and high-risk NASH are associated with complex metabolic derangements, signs of which can be detected in circulating blood. Multivariate models combining clinical (age, gender, diabetes, BMI), biochemical (liver enzymes, bilirubin, ferritin), and metabolic (HbA1c, HOMA-IR, blood lipids) factors have been investigated both for NASH diagnosis and risk stratification. Unfortunately, because many of the constituent variables are not liver-specific, these models are often confounded by other disease processes, such as inflammation and the metabolic syndrome. The FIB-4 is one of the most widely used serum biomarkers of advanced liver fibrosis. Its constituents include age, platelet count, AST, and ALT (aspartate and alanine aminotransferase), which are low cost and typically obtained as part of conventional liver care. FIB-4 has the advantage of low cost and wide availability, but as with most serum biomarkers can be confounded by many factors including age and presence of diabetes.

NAFLD can be subdivided into two forms: (1) non-alcoholic fatty liver (NAFL, ˜80%), which confers limited or no risk of cirrhosis, and (2) non-alcoholic steatohepatitis (NASH, ˜20%), a condition which confers substantial risk of progression to cirrhosis, in which hepatic fat accumulation is accompanied by inflammation and hepatocellular injury on liver biopsy. Recent work has shown liver fibrosis stage is a key predictor of ESLD in NASH, with moderate or greater liver fibrosis denoting particularly high risk of long-term liver specific mortality. The presence of moderate or greater liver fibrosis further increases progression risk and denotes high-risk NASH (hrNASH). NAFLD has also been shown to be a strong independent risk factor for cardiometabolic disease, with a markedly increased risk of cardiovascular morbidity and death.

NASH typically progresses over decades of repeated hepatic injury, healing, and fibrosis, culminating in cirrhosis, a state of severe and irreversible liver fibrosis. However, in the earlier stages, prior to cirrhosis, there is strong evidence that liver fat reduction is associated with decreased injury and fibrogenesis, arresting and even partially reversing liver fibrosis.

NAFLD prevalence is underestimated and progression is under recognized by primary care physicians. Accurate diagnosis is essential for optimal clinical care and also for recruitment for NAFLD therapeutics clinical trials. The current standard for diagnosis is a liver biopsy, which is invasive, costly, and is subject to sampling error, since the liver is not uniform and only 1/50,000 of the liver is sampled. As a result, only a small minority of NAFLD patients typically undergo liver biopsy. Biopsy is also unsuitable for screening asymptomatic at-risk individuals because of these same reasons, and because it varies across interpreters, and is not well-accepted by patients. Ultrasound shear-wave elastography (SWE) is an alternative, noninvasive and lower-cost approach, but currently is limited by inter-observer and intra-observer variability.

SWE imaging is accurate for the diagnosis decision support of cirrhosis and liver fibrosis. Previous study results reported SWE liver tissue stiffness measurements overlap between METAVIR fibrosis stages F1, F2, and to a lesser extent, F3, preventing clinically relevant liver fibrosis stage distinctions. This overlap was observed in all SWE studies, and is likely a consequence of disease biology rather than imperfect liver tissue stiffness measurement. When liver collagen is histopathologically quantified, similar overlap between fibrosis stages F1, F2, and to a lesser extent, F3, is observed. The observed overlap occurs because histopathologic liver fibrosis staging is based on the amount and anatomic distribution of excess collagen, whereas liver stiffness depends primarily on the amount of deposited collagen. This explains the weak correlation observed between liver fibrosis stage and SWE liver stiffness in many studies. This inherent limitation of liver stiffness measurements for liver fibrosis staging indicates a new method is needed to improve liver fibrosis staging with SWE. Since liver fibrosis is heterogeneously distributed, and this heterogeneity increases with liver fibrosis stage, 2D-SWE liver stiffness maps may contain additional information regarding liver fibrosis stage. This is particularly relevant in patients with early fibrosis, who may have regions of relatively normal liver tissue interspersed with regions of fibrosis leading to SWE sampling error and incorrect disease staging.

The majority of liver disease subjects may be left undiagnosed, without treatment, and at risk of progression, even though weight reduction is of benefit and numerous treatments may be available. The vast burden of undiagnosed disease implies therapeutics investment alone will not meaningfully alter the societal impact of NAFLD unless accompanied by widely deployed low-cost detection and risk stratification. Thus, there remains a need for less variable, more accurate, faster diagnosis of liver disease using SWE. There also remains a need for the same regarding staging liver disease with the ability to distinguish between overlapping stages.

### SUMMARY OF THE DISCLOSURE

The present disclosure addresses the aforementioned drawbacks by providing systems and methods for noninvasive staging and diagnosis decision support of diffuse liver disease from ultrasound shear-wave elastography. In some configurations, a multiparametric imaging analysis may be performed to extract features for liver disease diagnosis decision support or staging, such as for hrNASH. Deep learning may be used in combination with SWE feature extraction. In some configurations, SWE and US-based liver disease classification algorithms may be integrated with clinical and laboratory data in a disease prediction or staging model. Integration of statistical modeling of demographic and laboratory data with ultrasound imaging data in a machine learning model to produce a synthetic biomarker offers the potential to achieve superior classification accuracy with lower variability than either method alone, or with previous methods. This integration can be used to predict disease risk.

In one configuration, a method is provided for disease diagnostic decision support. The method includes accessing with a computer system, elastography data acquired from a subject. The method also includes selecting a region of interest (ROI) in the elastography data using the computer system, where the ROI is selected by implementing an automated algorithm with the computer system in order to minimize variability. Features may be extracted from the selected ROI using a statistical classifier implemented with a hardware processor and a memory of the computer system and a report may be generated for a user with a diagnostic decision support of a disease based upon the statistical classifier extracted features.

In one configuration, a system is provided for disease diagnostic decision support. The system includes a computer system configured to: i) access, with a computer system, elastography data acquired from a subject; ii) select a region of interest (ROI) in the elastography data using the computer system, where the ROI is selected by implementing an automated algorithm with the computer system in order to minimize variability; iii) extract features from the selected ROI using a statistical classifier implemented with a hardware processor and a memory of the computer system; and iv) generate a report for a user with a diagnostic decision support of a disease based upon the statistical classifier extracted features

In one configuration, a method is provided for constructing and implementing a trained machine learning algorithm in order to generate, from shear wave elastography data, a feature map that depicts spatial patterns of a liver disease staging. The steps of the method include constructing a trained machine learning algorithm by: i)accessing training data with a computer system, the training data comprising shear wave elastography (SWE) data and at least one of clinical data or laboratory data obtained from a plurality of subjects; ii) training a machine learning algorithm based on the training data, where the machine learning algorithm is trained on the training data in order to localize regions associated with different liver disease stages. The steps of the method also include generating a feature map that depicts spatial patterns of liver disease staging in a subject by inputting SWE data acquired from that subject to the trained machine learning algorithm

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention.

### BRIEF DESCRIPTION OF THE DRAWINGS

FIG. 1 is a block diagram of an example computer system that can implement the methods described in the present disclosure.

FIG. 2 is a block diagram of an example ultrasound system that can implement the methods described in the present disclosure.

FIG. 3 is a block diagram of an example computer system from FIG. 1 that can implement the methods described in the present disclosure.

FIG. 4 is a flowchart depicting non-limiting example steps for a method according to the present disclosure.

FIG. 5 is a flowchart depicting non-limiting example steps for a method according to the present disclosure.

### DETAILED DESCRIPTION

Described here are systems and methods for objective, noninvasive staging of diffuse liver disease, and other diseases, from ultrasound shear wave elastography (“SWE”). A liver fibrosis staging SWE image analysis algorithm or toolkit may be used to detect the presence of NAFLD and, in some configurations, may integrate demographic data, clinical data, laboratory data, conventional sonographic data, SWE data, and the like, into a combined hrNASH diagnosis decision support model. A diagnosis decision support includes providing guidance to a user on the possible diagnosis for a disease, staging a disease, indicating the progression of a disease, generating feature maps depicting spatial distribution of a disease, and the like. In some configurations, advanced image processing methods are used to extract fibrosis pattern information from 2D-SWE and US image data and this information is used to augment stiffness-based liver fibrosis staging methods.

Combining predictive models that use clinical and laboratory data, including such data that may be based on widely available and inexpensive blood tests, with low cost liver-specific noninvasive imaging offers the possibility of improved disease detection accuracy, such as hrNASH detection accuracy, at low additional cost.

In one aspect, the methods described here provide real-time feedback to an operator to aid image acquisition in order to reduce the number of measurements required, such as by augmenting image data into a diagnostic decision support model. In another aspect, the methods described here provide for assessing image quality.

In another aspect, the methods described in the present disclosure include automatically selecting a region-of-interest (“ROI”) from an image based on image processing to exclude tissue non-uniformities, such as blood vessels and lesions.

In yet another aspect, the methods described in the present disclosure include scoring liver fibrosis based on a machine learning algorithm implemented with a hardware processor and a memory. As one example, the machine learning algorithm can be a neural network. In general, images obtained with ultrasound SWE are input to the machine learning algorithm. The output, an estimate of a clinically recognized liver fibrosis score, can provide important diagnostic information to a clinician.

The methods described in the present disclosure can be implemented with a commercial ultrasound SWE device. The methods can also be implemented with a computer system.

In some configurations, SWE may be implemented using conventional ultrasound devices. SWE employs acoustically induced shear waves to measure tissue stiffness. Two broad categories of SWE in clinical use include Point SWE (pSWE), in which a single excitation yields a point shear wave velocity estimate, and two-dimensional SWE (2D-SWE), in which multiple focused acoustic excitations yield a band-like shear wave propagating through tissue, which generates an anatomic tissue stiffness map. In conventional 2D-SWE, a sonographer acquires an image, selects a region of interest (ROI) within the SWE image box, and the ultrasound machine then provides a mean stiffness within the ROI. To mitigate variability, the sonographer may repeat this process a number of times, such as ten times, and then takes the median value. Distinct stiffness pattern changes in advanced liver fibrosis indicate image analysis may improve SWE accuracy and reduce variability. The systems and methods of the present disclosure improve SWE accuracy for liver fibrosis staging.

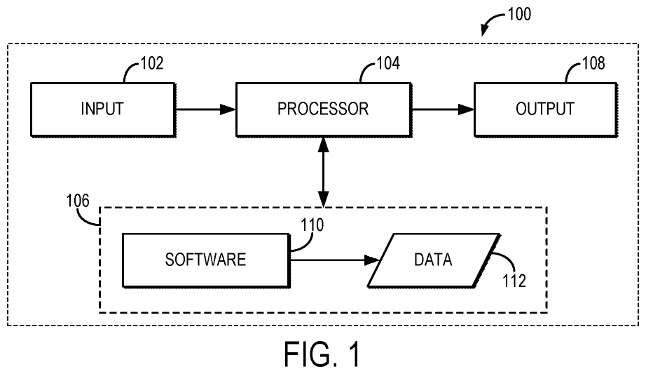

Referring now to FIG. 1, a block diagram of an example of a computer system 100 that can perform the methods described in the present disclosure is shown. The computer system 100 generally includes an input 102, at least one hardware processor 104, a memory 106, and an output 108. Thus, the computer system 100 is generally implemented with a hardware processor 104 and a memory 106.

In some embodiments, the computer system 100 can be a computer system integrated with an ultrasound device, such as an ultrasound SWE device. The computer system 100 may also be implemented, in some examples, by a workstation, a notebook computer, a tablet device, a mobile device, a multimedia device, a network server, a mainframe, one or more controllers, one or more microcontrollers, or any other general purpose or application-specific computing device.

The computer system 100 may operate autonomously or semi-autonomously, or may read executable software instructions from the memory 106 or a computer-readable medium (e.g., a hard drive, a CD-ROM, flash memory), or may receive instructions via the input 102 from a user, or any another source logically connected to a computer or device, such as another networked computer or server. Thus, in some embodiments, the computer system 100 can also include any suitable device for reading computer-readable storage media.

In general, the computer system 100 is programmed or otherwise configured to implement the methods and algorithms described in the present disclosure. For instance, the computer system 100 can be programmed to implement the methods descried in the present disclosure, such as by providing and implementing a suitable machine learning algorithm, which may be a neural network.

The input 102 may take any suitable shape or form, as desired, for operation of the computer system 100, including the ability for selecting, entering, or otherwise specifying parameters consistent with performing tasks, processing data, or operating the computer system 100. In some aspects, the input 102 may be configured to access or receive data, such as data acquired with an ultrasound device (e.g., ultrasound image data), which may be an ultrasound SWE device (e.g., SWE data). Such data may be processed as described in the present disclosure. In addition, the input 102 may also be configured to access or receive any other data or information considered useful for implementing the methods described in the present disclosure, such as clinical data, laboratory data, demographic data, and so on.

Among the processing tasks for operating the computer system 100, the one or more hardware processors 104 may also be configured to carry out any number of post-processing steps on data received by way of the input 102.

The memory 106 may contain software 110 and data 112, such as data acquired with an ultrasound device, and may be configured for storage and retrieval of processed information, instructions, and data to be processed by the one or more hardware processors 104. In some aspects, the software 110 may contain instructions directed to implementing the methods described in the present disclosure.

In addition, the output 108 may take any shape or form, as desired, and may be configured for displaying ultrasound images, mechanical property maps generated from ultrasound SWE data, and maps or other reports indicating liver fibrosis scores, in addition to other desired information.

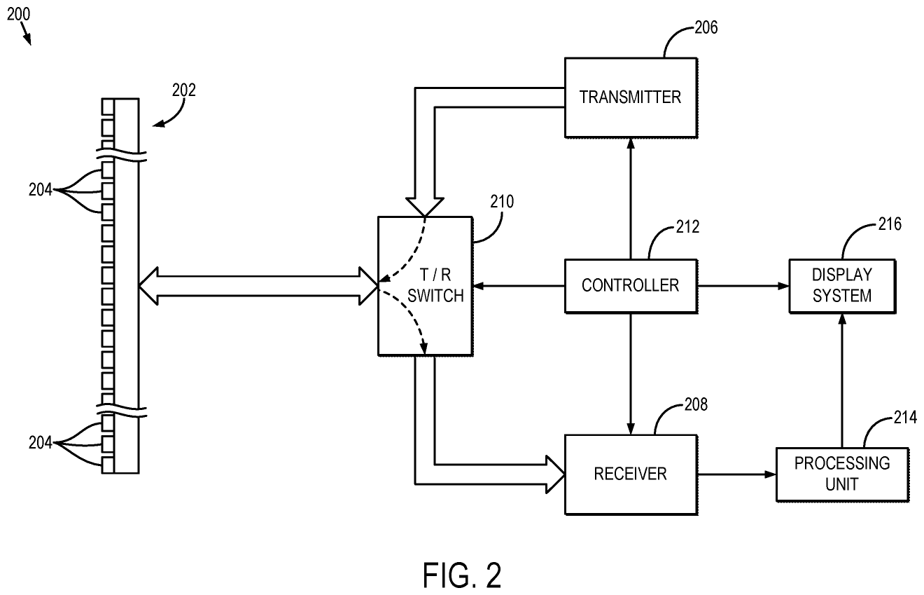

FIG. 2 illustrates an example of an ultrasound system 200 that can implement the methods described in the present disclosure. The ultrasound system 200 includes a transducer array 202 that includes a plurality of separately driven transducer elements 204. The transducer array 202 can include any suitable ultrasound transducer array, including linear arrays, curved arrays, phased arrays, and so on. Similarly, the transducer array 202 can include a 1D transducer, a 1.5D transducer, a 1.75D transducer, a 2D transducer, a 3D transducer, and so on.

When energized by a transmitter 206, a given transducer element 204 produces a burst of ultrasonic energy. The ultrasonic energy reflected back to the transducer array 202 (e.g., an echo) from the object or subject under study is converted to an electrical signal (e.g., an echo signal) by each transducer element 204 and can be applied separately to a receiver 208 through a set of switches 210. The transmitter 206, receiver 208, and switches 210 are operated under the control of a controller 212, which may include one or more processors. As one example, the controller 212 can include a computer system.

The transmitter 206 can be programmed to transmit unfocused or focused ultrasound waves. In some configurations, the transmitter 206 can also be programmed to transmit diverged waves, spherical waves, cylindrical waves, plane waves, or combinations thereof. Furthermore, the transmitter 206 can be programmed to transmit spatially or temporally encoded pulses.

The receiver 208 can be programmed to implement a suitable detection sequence for the imaging task at hand. In some embodiments, the detection sequence can include one or more of line-by-line scanning, compounding plane wave imaging, synthetic aperture imaging, and compounding diverging beam imaging.

In some configurations, the transmitter 206 and the receiver 208 can be programmed to implement a high frame rate. For instance, a frame rate associated with an acquisition pulse repetition frequency (“PRF”) of at least 100 Hz can be implemented. In some configurations, the ultrasound system 200 can sample and store at least one hundred ensembles of echo signals in the temporal direction.

The controller 212 can be programmed to design or otherwise implement an imaging sequence as known in the art. In some embodiments, the controller 212 receives user inputs defining various factors used in the design of the imaging sequence.

A scan can be performed by setting the switches 210 to their transmit position, thereby directing the transmitter 206 to be turned on momentarily to energize transducer elements 204 during a single transmission event according to the prescribed imaging sequence. The switches 210 can then be set to their receive position and the subsequent echo signals produced by the transducer elements 204 in response to one or more detected echoes are measured and applied to the receiver 208. The separate echo signals from the transducer elements 204 can be combined in the receiver 208 to produce a single echo signal.

The echo signals are communicated to a processing unit 214, which may be implemented by a hardware processor and memory, to process echo signals or images generated from echo signals. As an example, the processing unit 214 can implement the methods described in the present disclosure. Images produced from the echo signals by the processing unit 214 can be displayed on a display system 216.

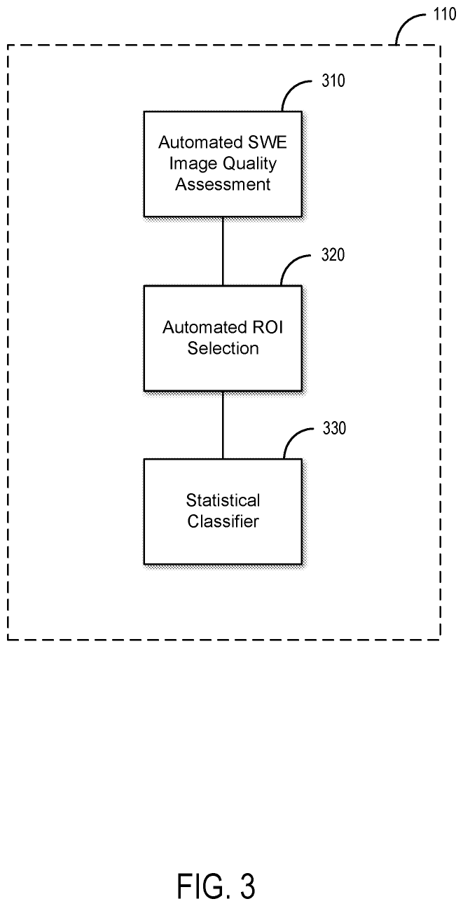

Referring to FIG. 3 in light of FIG. 1, software 110 may include a SWE-Assist toolkit, including: an automated SWE image quality assessment tool 310, an automated ROI Selection tool 320, and a statistical classifier 330, which may be used for stages greater than or equal to F2 fibrosis. In one non-limiting example, the statistical classifier 330 may be implemented with a classifier created by training with handcrafted features. In one non-limiting example, deep learning may be used to create the classifier in which features are automatically extracted prior to training. Features may include those described in Table 1, such as basic stiffness measures (e.g., estimated Young's Modulus (eYM) mean, minimum, maximum, and SD), and additional statistical features (eYM skewness, kurtosis, and entropy). In one non-limiting example, these features may be evaluated with a random forest classifier and support vector machine (SVM) in conjunction with principal components analysis (PCA). In one non-limiting example, a deep learning convolutional neural network (CNN) may be used, which is an approach well-suited to extracting complex, difficult to quantify image features.

TABLE 1
Summary of Image Features

| Image | Features | Purpose |
|---|---|---|
| SWE | Basic stiffness measures: | Fibrosis staging |
| | eYM, mean, min, max, standard | |
| | deviation | |
| | Additional statistics: eYM | |
| | skew, kurtosis, entropy | |
| | ROI histogram | |
| | CNN-based | |
| US | CNN-based detection of SWE | ROI selection |
| | artifacts: blood vessels, | |
| | calcification | |
| | Speckle, texture, attenuation | Liver fat content |
| | Skin to liver capsule distance | Associated with |
| | | NAFLD |

In one configuration, an automated image quality assessment tool 310 for SWE is provided. Elastography-histopathology discordance may be observed and iterative analyses may be performed to identify where image removal would improve elastographic liver fibrosis staging. These analyses may permit empiric identification of specific image features associated with elastography-histopathology discordance. In one non-limiting example, a database of images associated with incorrect liver fibrosis stage assignment, such as a database of 2D-SWE images, may be generated.

In one configuration, the image quality assessment may include determining a Percentage of Color Fill-In (PCFI) metric, computed as the ratio of the valid pixel number (pixels where a stiffness measurement exists) to the pixel size of the SWE box. In a typical “high quality” SWE image seen with elastography-histopathology concordance, the SWE image box is near-completely filled with color-coded pixels reflecting stiffness of the tissue, or SWE wave speed. Conversely, a typical “low quality” SWE image is where the SWE image box contains relatively few valid pixels and therefore less data. In current clinical practice, low quality measurements are common. This is currently addressed clinically by taking ten measurements in succession, which is the standard recommended number, and then taking the median of these measurements. Using an automated image selection algorithm in accordance with the present disclosure, a SWE image acceptance criterion with higher PCFI may be achieved. In one non-limiting example, a PCFI greater than 70% may be achieved with the same AUC of 0.74 for a METAVIR fibrosis stage greater than F2 diagnosis using a single image in the analysis. Automated image quality assessment via a PCFI threshold criterion method allows for improved measurement quality and/or reducing the required measurement number.

In conventional 2DSWE, the operator places a circular region of interest (ROI) in the SWE image box to obtain estimated Young's modulus (eYM) stiffness values. The goal is to avoid blood vessels, scars, calcifications and other potential sources of erroneous liver tissue stiffness measurement. The operator-placed ROI is small compared with the SWE image box and therefore makes use of only a minority of the available information. Moreover, ROI placement is highly operator-dependent, leading to inconsistent quality.

In one configuration, an automated ROI selection tool 320 is provided. An automated SWE data quality measurement and acquisition procedure may include automated serial measurement acquisition via “variability minimization” rectangular ROI raster scan. A rectangular ROI of predefined size (e.g. 16×16 pixels) may be raster scanned over the SWE image box, with repeated computation of inter-pixel variability measured as the standard deviation within the ROI. The lowest variability rectangular ROI may be selected. Low variability may be used as a quality measure based on empiric findings of lower inter-pixel variability being associated with better elastography-histopathology concordance. In some configurations, since liver heterogeneity increases with fibrosis stage and is particularly marked in cirrhosis, the variability-minimization ROI selection algorithm may be applied when mean eYM is below 8 kPa, which is below the eYM cutoff value for cirrhosis.

In one non-limiting example, an automated ROI following the above method may move to compensate for vessels that, if included in the measurement, may adjust the stiffness being reported. Whereas a manually selected ROI may be erroneously positioned near a vessel, which may result in an incorrectly high liver stiffness measurement, the algorithm-selected ROI may be at a more desirable location, away from the vessel.

In some configurations, image features may be extracted from both SWE and/or conventional US images using a statistical classifier 330. Features may be selected for being able to more accurately stage fibrosis in NAFLD patients. Diagnosis decision support, such as for aiding in the diagnosis of hrNASH and the like, may be improved by: (1) increasing the size and modifying the shape of the automated SWE image ROI, (2) developing clustering criteria for individual pixel inclusion (i.e., eliminating the concept of ROI and moving instead to “pixels of interest” (POI)), (3) developing additional SWE features across multiple images, and (4) developing features from the conventional B-mode US images.

To expand the ROI beyond a single fixed-size rectangle, arbitrary ROI shapes as well as multiple rectangular ROIs may be serially evaluated. Data-driven ROI selection criteria may be used to determine optimal ROI shapes, size, and acceptance criteria for hrNASH diagnosis, such as by using expert radiologist clinical knowledge.

For SWE images, basic stiffness measures and additional statistics may form a baseline feature set. To capture additional information, ROI/POI histograms may be used as features. The number of bins required may be determined for each image, or may be pre-selected to be on the order of a few tens of bins, such as in a range of 1-100 bins. Principal component analysis (PCA) may be performed to reduce total feature vector size and the risk of overfitting to the training data. Features may be developed for more than one image per case, including statistical features for each image, and features measured across all pixels in all images.

In some configurations, CNNs may be used both to learn features and for development of the disease diagnosis decision support tool, such as for aiding in the diagnosis of hrNASH. To operate CNNs on irregularly shaped POIs, zero padding may be applied to achieve a consistent dimension, and POI delineation may be constrained based on the CNN receptive field. In one non-limiting example, for a 16×16 receptive field, the POIs may be defined based as piecewise combinations of that field size.

Features may be extracted to (1) identify known sources of SWE artifacts (e.g., blood vessels), (2) estimate liver fat, (3) measure subcutaneous fat, and the like. Artifact detectors may be trained using CNNs based on images annotated by radiologists. The number of layers of weights (such as either shallow or deep) may be based on the data. If a deep network is required, transfer learning may be applied to capture low-level feature sets from pre-trained networks (such as AlexNet, VGGNet) to reduce required training set size. In some instances, transfer learning can begin with an existing network (e.g., an ImageNet network) trained on a large image number. In these instances, the final, fully connected layers at the end of such a network may be then be replaced and trained with application-specific data. Although the purpose of detecting SWE artifacts is to support ROI/POI selection, these may also prove valuable in future work as image registration landmarks for repeatable location selection in longitudinal monitoring.

Liver fat content may be reflected on US images as increased echogenicity and beam attenuation. A CNN-based model may be used to estimate steatosis using annotated images and informed by reference standard NASH-CRN histopathologic staging. Texture-based features may be measured with gray-level co-occurrence matrices (GLCMs), which reveals spatial relationship of pixels, and wavelet based techniques, which reveals information in the frequency domain.

Subcutaneous fat thickness may be associated with obesity and NAFLD. To measure this, a trained CNN may be used based on Radiologist annotation to detect the liver capsule, and may then be used to measure skin surface to liver capsule distance. Table 1 lists image features that may be extracted.

Table 2 summarizes a non-limiting example of a four-layer CNN configuration that may be used with the present disclosure containing two sets of 2D convolution and max pooling layers, followed by a dropout layer, and a fully connected layer trained to output a binary classification (0 for <F2 and 1 for ≥F2 fibrosis). The purpose of the dropout layer is to minimize overfitting.

TABLE 2
Four Layer CNN

| Layers | Filters |
|---|---|
| Conv2D (Size = 3 × 3, Stride = 1, Nonlinearity = ReLu) | 16 |
| Max pooling (Size = 2 × 2, Stride = 1) | 16 |
| Conv2D (Size = 3 × 3, Stride = 1, Nonlinearity = ReLu) | 32 |
| Max pooling (Size = 2 × 2, Stride = 1) | 32 |
| Dropout (Thresh = 0.5) | — |
| Fully connected (Output = 2) | — |

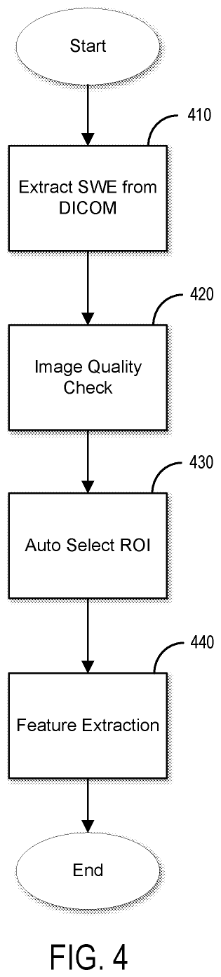

Referring to FIG. 4, non-limiting examples steps for a computer implemented method, such as with the SWE-Assist system described above, to provide disease diagnosis decision support, or staging, are shown. A SWE-Assist system as described above may be used to automate variability reduction and to maximize accuracy. Non-limiting examples may include extracting SWE data at step 410. In some configurations, this may include extracting data from the DICOM image header. Automatically assessing image quality may be performed at step 420. Quality criteria or thresholds may be determined by a user based upon the disease being diagnosed. In one non-limiting example, all images with PCFI <70% may be rejected as not meeting the quality criteria. Automatically selecting an ROI may be performed at step 430. Similarly as with the image quality, ROI parameters may be determined by a user based upon the disease to be diagnosed. In one non-limiting example, if the SWE image box has a mean pixel value <8 kPa, the rectangular auto-ROI is raster-scanned and auto-positioned to minimize variability. Feature extraction may be performed at step 440. In some configurations, feature extraction may use a statistical classifier. In one non-limiting example, the classifier is run to determine stages above F2 using the accepted 2D-SWE images and automatically generated elastography values.

In some configurations, features may be extracted from a rectangular ROI. SWE extracted features, such as stiffness features or data, elastography values, additional statistics histogram, and the like may be evaluated with random forest, SVM, and fully connected neural network classifiers. In addition, a CNN may be trained on the ROI pixel values. The value of US features may be assessed by concatenating these with extracted SWE features or through fully connected weights in the CNN. In some configurations, models may be used with arbitrary-shaped POIs. In some configurations, classifier performance may be increased through the use of larger data sets and adjusting class weights.

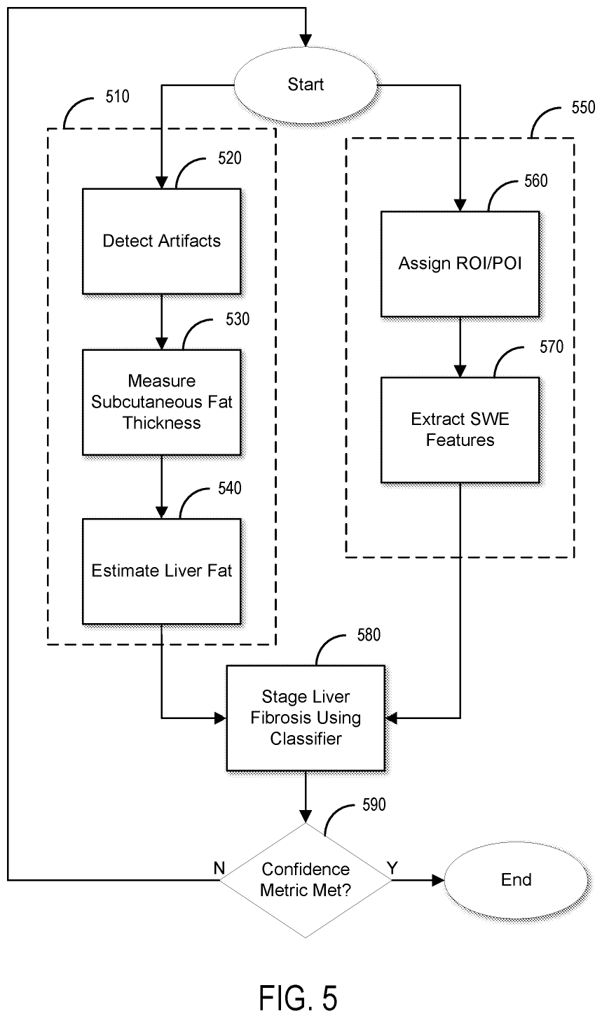

Referring to FIG. 5, non-limiting example steps for a method for liver disease diagnosis decision support are shown. In one non-limiting example, the liver disease diagnosis may be directed towards hrNASH detection. Method steps group 510 may be performed on an ultrasound image, and may include detecting artifacts at step 520, measuring subcutaneous fat thickness at 530, and estimating liver fat at step 540. Method steps group 550 may be performed on a SWE image and may include assigning ROI/POI at step 560 and extracting SWE features at step 570. Data may then be combined at step 580 for determining the stage of liver fibrosis, which may involve using a classifier as discussed above. A confidence metric may be assessed at step 590 where if a sufficient confidence level is not achieved by the results, then the process may be repeated. If the confidence level is achieved, then the process may end, such as by providing a display of the results to a user or otherwise providing a report of the results to a user.

In some configurations, multi-image modeling may be used in the diagnostic decision support method. A fixed number of images may be used, such as less than the current ten SWE images, or the number of images needed may be determined for a particular patient during processing. The multi-image classification process may start by determining if a single-image classifier output meets a predefined confidence level, which may be determined by prior empiric AUROC maximization at different proposed confidence levels. If it does, then the process will stop. If not, another US and SWE image pair may be classified and the classifier outputs combined. This process may be repeated until the desired confidence level is reached. Several approaches to combining classifier outputs may be used, such as (1) A standard m-out-of-n rule, where the combined classifier output is considered true if at least m out of n features specified in the rule are correct; (2) For classifier outputs that approximate probabilities, assuming statistical independence between images, computed scaled likelihoods can be combined through Bayes' Rule. Other methods for combining classifier outputs may be used. In some configurations, a confidence estimate provided by a Bayesian CNN may be assessed. Table 3 provides a summary of some non-limiting example machine learning algorithms that may be used with the present disclosure.

TABLE 3
Summary of Machine Learning Algorithms

| Image Number | Automated ROI/POI | | |
|---|---|---|---|
| Modelled | Selection | Features | Classifier |
| Single | Rectangular ROI | Basic | Random forest, |
| | Arbitrary-Shaped | statistics, | SVM, neural |
| | Pixels of Interest | histogram | network |
| | (POI) | +US features | |
| | | SWE pixel values | CNN |

| | | |
|---|---|---|
| Multiple | Arbitrary-Shaped | Optimal performance of above |
| | POI | with multi-image fusion |
| | | +operating point optimization |

CNN model accuracy may vary considerably depending on the network configuration (e.g., numbers of weight layers and nonlinearities, filter sizes at each layer, and the like). The CNN model structure may be optimized through many techniques. In some configurations, the sensitivity/specificity operating point of the CNN may be optimized to match clinical needs. In some configurations, a model that yields the highest AUROC is not necessarily the same as the model that yields the highest sensitivity at a particular desired false-positive rate, and so partial AUROCs may be used as an additional performance metric. A desired false-positive rate can be specified, and an optimal model based on the Neyman-Pearson criterion may be trained, employing training objective functions.

In some configurations, performance of the method may be evaluated. During a phased approach of building up the SWE/US multi-image modeling framework, performance metrics may be used, which include AUROC and sensitivity and specificity for liver disease, such as hrNASH, as a function of the mean and maximum number of images required per decision. Models may also be developed based on a training and validation set and tested with an independent test set. K-fold cross validation, for example, may be used to increase the measurement confidence of the performance metrics.

Prediction models may be tested when clinical and laboratory data are combined with conventional SWE. Non-limiting example clinical data that may be used is listed in Table 4 below.

TABLE 4
Clinical Data Examples

| Example Data | Example Source |
|---|---|
| Gender, ethnicity, age, BMI, medication use, | Medical record review |
| alcohol intake, comorbidities, signs and | |
| symptoms. | |
| Liver: AST, ALT, ALP, GGT, TB, IB, | Medical record review |
| DB, Albumin, | |
| Total proteins, PT. Other lab data: RBC, WBC, | |
| Platelets, glucose, creatinine, lipid profile, Hba1c | |
| Other liver-specific studies including viral | Medical record review |
| serology, ferritin, ceruloplasmin, | |
| AMA, ANA, AFP | |
| NASH-CRN scoring, METAVIR Score (if not | Blinded histopathology |
| NASH), NAS, steatosis score, Liver fibrosis stage | analysis |
| Liver stiffness (kPa), handcrafted features | SWE post-processing |
| Conventional (B-mode) US liver images | PACS |

In some configurations, clinical and laboratory data features, such as in Table 4, may be extracted together with conventional SWE, hrNASH-Det augmented SWE, and conventional B-mode ultrasound images to predict disease, such as hrNASH. In one non-limiting example, predictive variables may be identified using random forest methods. These methods use a two-stage strategy based on preliminary ranking of the explanatory variables using a permutation-based importance score followed by a stepwise forward strategy for variable introduction into the predictive model. This process reduces variable redundancy and minimizes overfitting. The selected variables may then be used to build a parsimonious risk prediction model using different machine learning classifier development algorithms. A non-limiting list of algorithms are listed in Table 5 below. As discussed above, the performance of each of the prediction algorithms may be tested, such as by using a multi-fold cross validation and the results may be used to select the algorithm that provides the highest AUROC.

TABLE 5
hrNASH Prediction Models

| Method | AUC |
|---|---|
| Linear discriminant analysis | 0.875 |
| Logistic regression | 0.875 |
| K-nearest neighbors | 0.792 |
| LogitBoost | 0.792 |
| Stochastic Boosting | 0.875 |
| Stepwise linear discriminant analysis | 0.667 |
| Support vector machine (SVM) | 0.875 |
| Random forest | 0.875 |
| Logistic model tree | 0.852 |

In one non-limiting example, in 116 subjects 27 variables were retrieved that are typically measured in standard NAFLD care. Based on these, a series of hrNASH prediction models in NAFLD patients were generated. The random forest method was used for variable selection to permit a parsimonious model for each prediction and to avoid overfitting. In this data set, predictive variable identification supported the hypothesis that the combination of SWE, clinical, and laboratory data may have additional value for hrNASH detection, yielding a combination of variable types for both outcomes. Nine different modelling methods were used to build fibrosis staging prediction models for NASH CRN fibrosis stages ≥F2, and ROC curves were plotted. 10-fold cross validation was used to minimize over-fitting. Table 5 lists non-limiting example modelling methods and their corresponding AUROC values for the present example. Findings across these methods were largely consistent, with AUROC for diagnosing ≥stage F2 fibrosis ranging from 0.667 for stepwise linear discriminant analysis to 0.875 for multiple models. The combination of SWE, clinical data, and laboratory data has predictive power for hrNASH. Further, SWE may add complementary information to standard blood test-based models used to detect advanced liver fibrosis.

In one example study, the methods described in the present disclosure were assessed with 3,329 SWE images from 328 subjects. The goal of this example study was to detect severe fibrosis or cirrhosis (stage F2 or above), as identified from a biopsy. An area under the receiver operating curve (“AUROC”) of 0.89 was achieved, which was a significant improvement over the standard SWE measurement approach of 0.74. Moreover, this result was achieved based on scoring a mean of 1.5 images, while the standard approached required a fixed number of 10 images, which take considerably longer to acquire. Further improvement (AUROC 0.93, sensitivity 95% and specificity 71%) was achieved based on scoring 4 images using a machine learning-based algorithm according to the present disclosure.

In another example study, the methods described in the present disclosure were assessed in 136 subjects undergoing liver biopsy. An AUROC of 0.77 for SWE, with sensitivity 91.4% and specificity 52.5% for diagnosis of METAVIR stage ≥F2 fibrosis at a cutoff of 7.29 kPa was shown. These results were validated in another 277 subjects, finding 95.4% sensitivity and 50.5% specificity for stage ≥F2 fibrosis at the same cutoff. Others have shown similar shear wave speed increases with hepatic fibrosis.

In some configurations, classifier overfit to the training data may be corrected through use of accepted techniques such as cross validation, and model validation in an independent data set. In some configurations, intermediate results may be displayed and high-to-low feature mappings may be used to aid in clinical utility.

In some configurations, pixel color maps of estimated tissue stiffness may be generated by the ultrasound device. Any shear wave elastography device may be used with the present disclosure, including other forms of generating tissue mechanical properties from tissue, such as a device for generating transient microelastography, comb-push shear elastography, vibro-acoustography, harmonic motion imaging, 3D quasistatic ultrasound elastography, and the like. In some configurations, transfer learning approaches may be used to minimize training time and complexity when transferring developed algorithms to other ultrasound platforms.

The present disclosure has described one or more preferred embodiments, and it should be appreciated that many equivalents, alternatives, variations, and modifications, aside from those expressly stated, are possible and within the scope of the invention.

1. A method for disease diagnostic decision support comprising:
a) accessing with a computer system, elastography data acquired from a subject;
b) selecting a region of interest (ROI) in the elastography data using the computer system, wherein the ROI is selected by implementing an automated algorithm with the computer system in order to minimize variability;
c) extracting features from the selected ROI using a statistical classifier implemented with a hardware processor and a memory of the computer system;
d) generating a report for a user with a diagnostic decision support of a disease based upon the statistical classifier extracted features.

2. The method of claim 1, wherein extracting features includes selecting features based upon at least one of: identifying sources of artifacts in the acquired data, identifying blood vessels in the selected region of interest, estimating fat content of a liver of the subject, or measuring subcutaneous fat of the subject.

3. The method of claim 1, wherein the extracted features include at least one of stiffness measures of an estimated Young's Modulus (eYM) mean, minimum, maximum, standard deviation, skewness, kurtosis, or entropy.

4. The method of claim 1, wherein a convolutional neural network is used at least for extracting image features, or for the statistical classifier.

5. The method of claim 1, wherein implementing the automated algorithm includes repeatedly computing an inter-pixel variability measured as a standard deviation within the ROI.

6. The method of claim 1, wherein the ROI includes one of a rectangular ROI, an arbitrarily shaped ROI, or a plurality of pixels of interest (POIs) and at least some of the POIs are not spatially connected.

7. (canceled)

8. (canceled)

9. (canceled)

10. (canceled)

11. The method of claim 1, wherein accessing the elastography data includes accessing shear wave elastography (SWE) data, and further comprising accessing ultrasound data acquired from the subject, wherein the ultrasound data are combined with the SWE data, and wherein the diagnostic decision support includes staging liver fibrosis for the subject.

12. (canceled)

13. (canceled)

14. A system for disease diagnostic decision support comprising:
a computer system configured to:
i) access, with a computer system, elastography data acquired from a subject;
ii) select a region of interest (ROI) in the elastography data using the computer system, wherein the ROI is selected by implementing an automated algorithm with the computer system in order to minimize variability;
iii) extract features from the selected ROI using a statistical classifier implemented with a hardware processor and a memory of the computer system;
iv) generate a report for a user with a diagnostic decision support of a disease based upon the statistical classifier extracted features.

15. The system of claim 14, wherein the computer system is configured to extract features that include selecting features based upon at least one of: identifying sources of artifacts in the acquired data, identifying blood vessels in the selected region of interest, estimating fat content of a liver of the subject, or measuring subcutaneous fat of the subject.

16. The system of claim 14, wherein the computer system is configured to extract features that include at least one of stiffness measures of an estimated Young's Modulus (eYM) mean, minimum, maximum, standard deviation, skewness, kurtosis, or entropy.

17. (canceled)

18. The system of claim 14, the computer system is configured to repeatedly compute an inter-pixel variability measured as a standard deviation within the ROI.

19. (canceled)

20. (canceled)

21. (canceled)

22. (canceled)

23. (canceled)

24. The system of claim 14, wherein the computer system is configured to access shear wave elastography (SWE) data and access ultrasound data acquired from the subject, wherein the ultrasound data are combined with the SWE data, and wherein the diagnostic decision support includes staging liver fibrosis for the subject.

25. (canceled)

26. (canceled)

27. A method for constructing and implementing a trained machine learning algorithm in order to generate, from shear wave elastography data, a feature map that depicts spatial patterns of a liver disease staging, the steps of the method comprising:
constructing a trained machine learning algorithm by:
(i) accessing training data with a computer system, the training data comprising shear wave elastography (SWE) data and at least one of clinical data or laboratory data obtained from a plurality of subjects;
(ii) training a machine learning algorithm based on the training data, wherein the machine learning algorithm is trained on the training data in order to localize regions associated with different liver disease stages;
generating a feature map that depicts spatial patterns of liver disease staging in a subject by inputting SWE data acquired from that subject to the trained machine learning algorithm.

28. The method as recited in claim 27, wherein the training data further comprise image features derived from the SWE data.

29. The method as recited in claim 28, wherein the image features include at least one of stiffness measures of an estimated Young's Modulus (eYM) mean, minimum, maximum, standard deviation, skewness, kurtosis, or entropy.

30. The method as recited in claim 27, wherein a region-of-interest is selected in the SWE data and only the SWE data contained in the ROI are input to the trained machine learning algorithm in order to generate the feature map.

31. The method as recited in claim 30, wherein the ROI is one of a rectangular ROI or an arbitrarily shaped ROI.

32. (canceled)

33. The method as recited in claim 30, wherein the ROI comprises a plurality of pixels of interest (POIs) and at least some of the POIs are not spatially connected.

34. The method as recited in claim 30, wherein the ROI is selected by constructing a machine learning algorithm that is trained on training data comprising ultrasound image data in order to localize an ROI.
